(19) United States
(12) Patent Application Publication
(10) Pub. No.: US 2008/0229520 A1
(43) Pub. Date: Sep. 25, 2008

(54) Hair Colorants with Indigoid Vat Dyes

(76) Inventors: Manuela Javet, Marly (CH); Catherine Mueller, Marly (CH); Anita Roulin, Villarlod (CH)

Correspondence Address: MICHAEL J. STRIKER, 103 EAST NECK ROAD, HUNTINGTON, NY, 11743, US

(21) Appl. No.: 10/592,825

(22) PCT Filed: Nov. 24, 2004
(86) PCT No.: PCT/EP04/13306

§ 371 Date: Sep. 14, 2006

(30) Foreign Application Priority Data
Mar. 26, 2004 (DE) 10 2004 014 763.9

(51) Int. Cl. A61K 8/72; A61Q 5/10

(52) U.S. Cl. 8/405

(57) ABSTRACT

The agent for coloring hair contains at least one indigoid vat dye, at least one cationic compound, and as reducing agent at least one compound which in a strongly alkaline medium forms an enediol. In the method of coloring hair the pre-reduced indigoid vat dye and the cationic compound are applied to the hair and allowed to act on the hair at a pH of 4 to 11, preferably under protection from atmospheric oxygen. Then the vat dye is re-oxidized with atmospheric oxygen or an oxidant, such as hydrogen peroxide.

The present invention relates to an agent and a method for coloring hair, particularly human hair, with so-called indigoid vat dyes.

Although indigoid vat dyes are widely used for dyeing cellulosic fibrous materials, they are very rarely used for animal fibers, for example wool or hair, because these fibers do not withstand the required very high pH of about 13 without undergoing drastic damage. Moreover, in most cases very aggressive reducing agents are used, for example sodium dithionite (hydrosulfite) or sodium hydrogen sulfite. For these reasons, dyeing of animal fibers with vat dyes has thus far not been adopted in practice.

In the dyeing of textiles, attempts have already been made to replace hydrosulfites with other reducing agents as vatting agents also because hydrosulfites cause considerable contamination of wastewater. In EP 0 357 548 A1, enediols have been proposed as an appropriate replacement because under alkaline conditions they reduce the dyes practically as well as does dithionite, but are much better tolerated environmentally. These methods, however, can only be applied to cellulosic fibers and not to keratin ones, because a pH of about 13 is still required for the dyeing. Alternatively, for hair dyeing it has been proposed in DE 44 27 888 A1 to reduce vat dyes by use of laser light. Because of the use of laser radiation, however, this method is hardly applicable in practice.

The goal was therefore to provide a method whereby hair can be dyed with vat dyes in simple and gentle manner.

Surprisingly, we have now found that dyeing with vat dyes (for example indigo) can also be carried out under physiologically tolerable conditions if a cationic compound is added to the dye mixture and the pH is appropriately adjusted before the dyeing. In this manner, intense uniform hair colorations are obtained while at the same time the hair is treated as gentle as possible. Moreover, this method allows brightening dyeing to be achieved with partial or complete decolorization of the natural pigment melanin during the final oxidation step.

The present invention therefore has for an object an agent for coloring hair, characterized in that it contains at least one indigoid vat dye, at least one cationic compound and as reducing agent at least one compound which in an alkaline medium forms an enediol.

The present invention also has for an object a ready-to-use agent for coloring hair, characterized in that it contains a reduced indigoid vat dye formed in the presence of a cationic compound by the action of a compound which in an alkaline medium forms an enediol, said agent having a pH of 4 to 11.

By indigoid vat dyes in the sense of the invention are meant, in particular, indigo and indirubin (C.I. 75790) as well as indigo derivatives, for example 6,6′-dibromoindigo, 5,5′,7,7′-tetrabromoindigo, 4,4′,7,7′-tetrachloroindigo, thioindigo, 6,6′-dichloro-4,4′-dimethylthioindigo, 5,5′-dichloro-7,7′-dimethylthioindigo, 4,4′,7,7′-tetramethylthioindigo and mixtures thereof. Other suitable vat dyes from this group can be found in the Color Index (C.I.), third edition, 1971, published by the Society of Dyers and Colorists, in the sections on vat dyes and sulfur dyes.

The indigoid vat dyes are used in a total amount from 0.01 to 10 wt. % and preferably from 0.1 to 2 wt. %.

The reducing agents used are compounds which in an alkaline medium form enediols (endiolates), for example monohydroxyacetone, dihydroxyacetone, acetoin, glutaroin, adipoin, glycol aldehyde, benzoin, 2,3-dihydroxyacrylaldehyde and cyclopentadiolone, among which acetoin, monohydroxyacetone and dihydroxyacetone are particularly preferred.

Suitable alkalinizing agents for the reduction step are sodium or potassium hydroxide, or the alkali metal salts of aromatic monohydroxy- or polyhydroxy compounds, for example sodium or potassium phenoxide and sodium or potassium cresolate. The reduction step is generally carried out at a pH of 10 to 13.

The reducing agent is used either in an equimolar amount or in an up to 50-fold molar excess, based on the vat dye, a 3-fold to 5-fold molar excess being particularly preferred.

Moreover, the reduction can be carried out in the presence of an organic solvent which is used at a concentration from 0.1 to 80 weight percent and preferably from 5 to 50 weight percent. The organic solvents should form a homogeneous phase with water. Suitable are both protic and aprotic organic solvents, for example ethanol, n-propanol, isopropanol, n-butanol, glycols, for example ethylene glycol and propylene glycol, furthermore lactones, lactams and amides of the lower carboxylic acids as well as ureas, sulfones and sulfoxides. Mixtures of said solvents can also be used.

To accelerate the reduction and to improve the absorption of the dye by the fiber, cationic compounds can be used, preferably cationic polymers such as, for example Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium-44, Polyquaternium-46, Polyquaternium-47, Polyquaternium-51, Polyquaternium-55, Polyquaternium-57, hydroxypropylguar-hydroxypropyltrimethylammonium chloride, Quaternium-80, guar-hydroxypropyltrimethylammonium chloride or mixtures thereof. Particularly preferred are Polyquaternium-4 (hydroxyethylcellulose-dimethyldiallylammonium chloride copolymer), Polyquaternium-7 (dimethyldiallylammonium chloride-acrylamide copolymer) and Polyquaternium-22 (acrylic acid-diallyldimethylammonium chloride co-polymer).

The cationic compounds are used in a total amount from 0.001 to 5 weight percent and particularly from 0.1 to 1 weight percent.

For the dyeing that follows the reduction step, we found it to be advantageous to add ammonia or ammonium ions, for example in the form of aqueous ammonia or ammonium salts. Amines such as, for example, glucosamines, aminomethylpropanol, monoethanolamine and triethanolamine, or inorganic bases, for example ammonium carbonate, ammonium hydrogen carbonate and ammonium carbonate [sic] can also be added.

After the reduction, the strongly alkaline pH of the dye composition is adjusted to a physiologically tolerable value of 4 to 11 and preferably 6 to 10 by use of an acid or of a hydrolyzable substance, for example a salt or an ester. The adjustment to the desired pH can be carried out by use of, for example, buffer systems or of inorganic or organic acids, for example lactic, citric, tartaric, glycolic, acetic or phosphoric acid or with ammonium sulfate, ammonium chloride or cetyl lactate.

After the reduction step, common oxidation dye precursors can be added to the colorant of the invention. Suitable oxidation dye precursors are, for example, the following developers, couplers and self-coupling compounds:

(i) Developers: 1,4-diaminobenzene (p-phenylenediamine), 1,4-diamino-2-methylbenzene (p-toluylenediamine), 1,4-diamino-2,6-dimethylbenzene, 1,4-diamino-3,5-di-ethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)-benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[(2-acetylamino)ethoxy]-1,4-diaminobenzene, 4-phenylaminoaniline, 4-dimethylaminoaniline, 4-diethylaminoaniline, 4-dipropylaminoaniline, 4-[ethyl(2-hydroxyethyl)amino]aniline, 4-[di(2-hydroxyethyl)-amino]aniline, 4-[di(2-hydroxyethyl)amino-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(2-hydroxyethyl)benzene, 1,4-diamino-2-(1-methylethyl)benzene, 1,3-bis[(4-amino-phenyl)(2-hydroxyethyl)amino]-2-propanol, 1,4-bis[(4-aminophenyl)amino]butane, 1,8-bis-(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol and 2-amino-5-methylphenol, alone or in admixture with one another.

(ii) Couplers: N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxy-ethoxy)-5-methylbenzene, 2,4-di[(2-hydroxyethyl)amino]-1,5-dimethoxybenzene, 2,3-di-amino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 2,4-diamino-(di(2-hydroxyethoxy]benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethyl-aminophenol, 3-diethylaminophenol, 5-amino-2-methylphenol, 5-amino-4-fluoro-2-methyl-phenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5[(2-hydroxyethyl)amino]-4-methoxy-2-methylphenol, 5-[(2-hydroxyethyl)amino]-2-methylphenol, 3[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxy-propyl)amino]-2-methylphenol, 3-[(2,3-dihydroxypropyl)amino]-2-methylphenol, 3-[(2-hydroxyethyl)amino]-2-methylphenol; 2-amino-3-hydroxypyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxy-benzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxy-aniline, 5-[(2-hydroxyethyl)amino]-1,3-benzodioxol, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione, alone or in admixture with one another.

(iii) Self-coupling compounds: 2-amino-5-methylphenol, 2-amino-6-methylphenol, 2-amino-5-ethoxyphenol and 2-propylamino-5-aminopyridine.

The agent of the invention contains the oxidation dye precursors in a total amount from about 0.01 to 12 weight percent and particularly from about 0.2 to 6 weight percent.

For the purpose of attaining certain color shades, the colorant can also contain common natural and/or synthetic direct dyes, for example vegetable dyes such as, for example, henna, furthermore triphenylmethane dyes, aromatic nitro dyes, azo dyes, quinone dyes and cationic or anionic dyes.

Suitable synthetic dyes are, for example 1,4-bis[(2-hydroxyethyl)amino]-2-nitrobenzene, 1-(2-hydroxyethyl)amino-2-nitro-4-[di(2-hydroxyethyl)amino]benzene (HC Blue No. 2), 1-amino-3-methyl-4-[(2-hydroxyethyl)amino]-6-nitrobenzene (HC Violet No. 1), 4-[ethyl-(2-hydroxyethyl)amino]-1-[(2-hydroxyethyl)amino]-2-nitrobenzene hydrochloride (HC Blue No. 12), 4-[di(2-hydroxyethyl)amino]-1-[(2-methoxyethyl)amino-2-nitrobenzene (HC Blue No. 11), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitro-benzene hydrochloride (HC Blue No. 9), 1-(3-hydroxypropylamino)-4-[di(2-hydroxy-ethyl)amino]-2-nitrobenzene (HC Violet No. 2), 1-methylamino-4-[methyl-(2,3-dihydroxy-propyl)amino-2-nitrobenzene (HC Blue No. 6), 2-[(4-amino-2-nitrophenyl)amino]-5-dimethylaminobenzoic acid (HC Blue No. 13), 1-amino-4-[(2-hydroxyethyl)amino]-2-nitrobenzene (HC Red No. 7), 2-amino-4,6-dinitrophenol, 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxy-ethyl)amino]-2-nitrobenzene (HC Red No. 3), 4-amino-3-nitrophenol, 4-[(2-hydroxyethyl)amino]-3-nitrophenol, 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro-4-[(2,3-dihydroxypropyl)amino]-2-nitrobenzene (HC Red No. 10), 5-chloro-1,4-[di-(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1-amino-2-[(2-hydroxyethyl)amino]-5-nitrobenzene (HC Yellow No. 5), 1-(2-hydroxyethoxy)-2[(2-hydroxyethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitrophenol, 1-(2-hydroxyethoxy)-3-methylamino-4-nitrobenzene, 2,3-(dihydroxypropoxy)-3-methylamino-4-nitrobenzene, 2-[(2-hydroxyethyl)-amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene. 4-[(2,3-dihydroxypropyl)amino]-3-nitro-1-trifluoromethylbenzene (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene (HC Yellow No. 13), 4-[(2-hydroxyethyl)amino]-3-nitrobenzonitrile (HC Yellow No. 14), 4-[(2-hydroxyethyl)amino]-3-benzamide (HC Yellow No. 15), 1,4-di[(2,3-dihydroxypropyl)amino]-9,10-anthraquinone, 1-[(2-hydroxyethyl)amino]-4-methylamino-9,10-anthraquinone (C.I. 61505, Disperse Blue No. 3), 2-[(2-aminoethyl)amino]-9,10-anthraquinone (HC Orange No. 5), 1-hydroxy-4-[(4-methyl-2-sulfophenyl)amino]-9,10-anthraquinone, 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (C.I. 62015, Disperse Red No. 11, Solvent Violet No. 26), 1,4-dihydroxy-5,8-bis[(2-hydroxyethyl)amino]-9,10-anthraquinone (C.I. 62500, Disperse Blue No. 7, Solvent Blue No. 69), 9-(dimethylamino)benzo[a]phenoxazin-7-ium chloride (C.I. 51175; Basic Blue No. 6), di[4-(diethylamino)phenyl][4-(ethylamino)naphthyl]carbenium chloride (C.I. 42595; Basic Blue No. 7), 3,7-di(dimethylamino)phenothiazin-5-ium chloride (C.I. 52015; Basic Blue No. 9), di[4-(dimethylamino)phenyl][4-(phenylamino)naphthyl]carbenium chloride (C.I. 44045; Basic Blue No. 26), 2-[(4-(ethyl(2-hydroxyethyl)amino)phenyl)azo]-6-methoxy-3-methyl-benzothiazolium methylsulfate (C.I. 11154; Basic Blue No. 41), 8-amino-2-bromo-5-hydroxy-4-imino-6-{[3-(trimethylammonio)phenyl]amino}-1(4H)-naphthalenone chloride (C.I. 56059; Basic Blue No. 99), bis[4-(dimethylamino)phenyl][4-(methylamino)phenyl]carbenium chloride (C.I. 42535; Basic Violet No. 1), tris[4-(dimethylamino)phenyl]carbenium chloride (C.I. 42555; Basic Violet No. 3), 2-[3,6-(diethylamino)dibenzopyranium-9-yl]benzoyl chloride (C.I. 45170; Basic Violet No. 10), di(4-aminophenyl)(4-amino-3-methylphenyl)carbenium chloride (C.I. 42510; Basic Violet No. 14), 1,3-bis[(2,4-diamino-5-methylphenyl)azo]-3-methylbenzene (C.I. 21010; Basic Brown No. 4), 1-[(aminophenyl]-azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 1-[(4-amino-3-nitrophenyl)azo]-7-(trimethylammonio)-2-naphthol chloride (C.I. 12251; Basic Brown No. 17) [sic], 3,7-diamino-2,8-dimethyl-5-phenylphenazinium chloride (C.I. 50240; Basic Red No. 2), 1,4-dimethyl-5-{[4-(dimethylamino)phenyl]azo}-1,2,4-triazolium chloride (C.I. 11055; Basic Red No. 22), 2-hydroxy-1-[(2-methoxyphenyl)azo]-7-(trimethylammonio)naphthalene chloride (C.I. 12245; Basic Red No. 76), 2-{2-[(2,4-dimethoxyphenyl)amino]ethenyl}-1,3,3-trimethyl-3H-indol-1-ium chloride (C.I. 48055; Basic Yellow No. 11), 3-methyl-1-phenyl-4[(3-(trimethylammonio)phenyl]azo}pyrazol-5-one chloride (C.I. 12719; Basic Yellow No. 57), bis[4-((dimethylamino)phenyl]phenylcarbenium hydrogen sulfate (1:1) (C.I. 42040; Basic Green No. 1), 1-[di(2-hy-droxyethyl)amino]-3-methyl-4-[(4-nitrophenyl)azo]benzene (C.I. 11210; Disperse Red No. 17), 4-[(4-amino-phenyl)azo]-1-[di(2-hydroxyethyl)amino]-3-methylbenzene (HC Yellow No. 7), 2,6-diamino-3-[(pyridin-3-yl)azo]pyridine, 6-hydroxy-5-[(4-sulfophenyl)azo]-2-naphthalenesulfonic acid disodium salt (C.I. 15985; Food Yellow No. 3; FD&C Yellow No. 6), 2,4-dinitro-1-naphthol-7-sulfonic acid disodium salt (C.I. 10316; Acid Yellow No. 1; Food Yellow No. 1), 2-(indan-1,3-dion-2-yl)quinoline-x,x-sulfonic acid (mixture of mono- and disulfonic acid) (C.I. 47005; D&C Yellow No. 10; Food Yellow No. 13; Acid Yellow No. 3), 5-hydroxy-1-(4-sulfophenyl)-4-[(4-sulfophenyl)azo]pyrazole-3-carboxylic acid trisodium salt (C.I. 19140; Food Yellow No. 4; Acid Yellow No. 23), 9-(carboxyphenyl-6-hydroxy-3H-xanthen-3-one (C.I. 45350; Acid Yellow No. 73, D&C Yellow No. 8), 5[(2,4-dinitrophenyl)amino]-2-phenylaminobenzenesulfonic acid sodium salt (C.I. 10385; Acid Orange No. 3), 4-[(2,4-di-hydroxyphenyl)azo]benzenesulfonic acid monosodium salt (C.I. 14270; Acid Orange No. 6), 4-[(2-hydroxynaphth-1-yl)azo]benzenesulfonic acid sodium salt (C.I. 15510; Acid Orange No. 7), 4-[(2,4-dihydroxy-3-[(2,4-dimethylphenyl)azo]phenylazo]benzenesulfonic acid sodium salt (C.I. 20170; Acid Orange No. 24), 4-hydroxy-3-[(sulfonaphth-1-yl)azo]-1-naphthalenesulfonic acid disodium salt (C.I. 14720; Acid Red No. 14, 6-hydroxy-5-[(4-sulfonaphth-1-yl)azo]-2,4-naphthalenedisulfonic acid trisodium salt (C.I. 16255; Ponceau 4R; Acid Red No. 18), 3-hydroxy-4-[(4-sulfonaphth-1-yl)azo]-2,7-naphthalenedisulfonic acid trisodium salt (C.I. 16185; Acid Red No. 27), 8-amino-1-hydroxy-2-(phenylazo)-3,6-naphthalenedisulfonic acid disodium salt (C.I. 17200; Acid Red No. 33), 5-(acetylamino)-4-hydroxy-3-[(2-methylphenyl)azo]-2,7-naphthalenedisulfonic acid disodium salt (C.I. 18065; Acid Red No. 35), 2-(3-hydroxy-2,4,5,7-tetraiododibenzopyran-6-on-9-yl)benzoic acid disodium salt (C.I. 45430; Acid Red No. 51), N-[6-(diethylamino)-9-(2,4-disulfophenyl)-3H-xanthen-3-ylidene]-N-ethylethanammonium hydroxide, inner salt, sodium salt (C.I. 45100; Acid Red No. 52), 8-{[4-(phenylazo)phenyl]azo-7-naphthol-1,3-disulfonic acid disodium salt (C.I. 27290; Acid Red No. 73), 2′,4′,5′,7′-tetrabromo-3′,6′-dihydroxyspiro{isobenzofuran-1(3H),9′[9H]xanthen}-3-one disodium salt (C.I. 45380; Acid Red No. 87), 2′,4′,5′,7′-tetrabromo-4,5,6,7-tetrachloro-3′,6′-dihydroxyspiro{isobenzofuran-1-(3H),-9′[9H]-3-one disodium salt (C.I. 45410; Acid Red No. 92), 3′,6′-dihydroxy-4′,5′-diio-dospiro[1(3H),9′(9H)-xanthen]-3-one disodium salt (C.I. 45425; Acid Red No. 95), (2-sulfo-phenyl)di[4-(ethyl((4-sulfophenyl)methyl)amino)phenyl]carbenium disodium salt, betaine (C.I. 42090; Acid Blue No. 9; FD&C Blue No. 1), 1,4-bis[(2-sulfo-4-methylphenyl)amino]-9,10-anthraquinone disodium salt (C.I. 61570; Acid Green No. 25), bis[4-(di-methylamino)phenyl]-(3,7-disulfo-2-hydroxynaphth-1-yl)carbenium inner salt, monosodium salt (C.I. 44090; Food Green No. 1; Acid Green No. 50), bis[4-(diethylamino)-phenyl](2,4-disulfophenyl)carbenium inner salt, sodium salt (2:1) (C.I. 42045; Food Blue No. 3; Acid Blue No. 1), bis[4-(diethylamino)phenyl](5-hydroxy-2,4-disulfophenyl)carbenium inner salt, calcium salt (2:1) (C.I. 42051; Acid Blue No. 3), 1-amino-4-(cyclohexylamino)-9,10 anthraquinone-2-sulfonic acid sodium salt (C.I. 62045; Acid Blue No. 62, 2-(1,3-dihydro-3-keto-5-sulfo-2H-indol-2-ylidene)-2,3-dihydro-3-keto-1H-indol-5-sulfonic acid disodium salt (C.I. 73015), Acid Blue No. 74), 9-(2-carboxyphenyl)-3-[(2-methyl-phenyl)amino]-6-[(2-methyl-4-sulfophenyl)amino]xanthylium inner salt, monosodium salt (C.I. 45190; Acid Violet No. 9), 1-hydroxy-4-[(4-methyl-2-sulfo-phenyl)amino]-9,10-anthraquinone sodium salt (C.I. 60730; D&C Violet No. 2, D&C Violet No. 43), bis{3-nitro-4-[(4-phenylamino)-3-sulfophenylamino]phenyl}sulfone (C.I. 10410; Acid Brown No. 13), 5-amino-4-hydroxy-6-[(4-nitrophenyl)azo]-3-(phenylazo)-2,7-naphthalenedisulfonic acid di-sodium salt (C.I. 20470; Acid Black No. 1), 3-hydroxy-4-[(2-hydroxynaphth-1-yl)azo]-7-nitro-1-naphthalenesulfonic acid chromium complex (3:2) (C.I. 15711; Acid Black No. 52), 3-[(2,4-dimethyl-5-sulfophenyl)azo]-4-hydroxy-1-naphthalenesulfonic acid disodium salt (C.I. 14700; Food Red No. 1; FD&C Red No. 4), 4-(acetylamino)-5-hydroxy-6-[(7-sulfo-4-[(4-sulfophenyl)azo]naphth-1-yl)azo]-1,7-naphthalenedisulfonic acid tetrasodium salt (C.I. 28440; Food Black No. 1) and 3-hydroxy-4-(3-methyl-5-keto-1-phenyl-4,5-dihydro-1H-pyrazol-4-ylazo)naphthalene-1-sulfonic acid sodium salt, chromium complex (Acid Red No. 195), alone or in combination with one another.

The total amount of direct dyes in the agent of the invention is about 0.01 to 7 weight percent and preferably about 0.2 to 4 weight percent.

Other known and common dyes for hair dyeing that can be contained in the colorant of the invention are described in, among other publications, E. Sagarin, “Cosmetics, Science and Technology”, Interscience Publishers Inc., New York (1957), pages 503 ff, in H. Janistin, “Handbuch der Kosmetika und Riechstoffe” [Handbook of Cosmetics and Fragrances], vol. 3 (1973), pages 388 ff, and in K. Schrader, “Grundlagen und Rezepturen der Kosmetika” [Fundamentals and Formulations of Cosmetics], 2nd edition (1989), pages 782-815.

Moreover, the agent of the invention can contain antioxidants such as, for example, ascorbic acid, thioglycolic acid and sodium sulfite as well as agents for complexing heavy metals, for example an ethylenediamine tetraacetate or nitriloacetic acid, in an amount of up to about 0.5 weight percent. Perfume oils can be contained in the dye carrier composition of the invention in an amount of up to about 1 weight percent. Naturally, the afore-described hair colorant can optionally contain other additives commonly used in hair colorants, for example thickeners, for example homopolymers of acrylic acid, vegetable gums, cellulose derivatives and starch derivatives, algae polysaccharides, amphiphilic associative thickeners, furthermore preservatives,; antioxidants, for example sodium sulfite, thioglycolic acid or ascorbic acid; complexing agents; solvents such as water or the afore-described lower aliphatic alcohols, for example aliphatic alcohols with 1 to 4 carbon atoms, such as ethanol, pro-panol and isopropanol, or glycols such as glycerol and 1,2-propylene glycol, or sorbitol, and wetting agents, or emulsifiers from the classes of anionic, cationic, amphoteric or nonionic surface-active substances and furthermore softeners; vaselines, silicone oils, paraffin oil, polysorbates and fatty acids as well as hair-care agents, such as the cationic polymers or resins, lanolin derivatives, cholesterol, vitamins, pantothenic acid, betaine and salts, such as sodium chloride and sodium sulfate. The said constituents are used in amounts commonly employed for such purposes, for example the wetting agents and emulsifiers at a concentration from 0.1 to 30 weight percent and the hair-care agents at a concentration from 0.1 to 5.0 weight percent.

The agent of the invention is preferably produced in the form of an aqueous or aqueous-alcoholic preparation, for example as a thickened solution, emulsion, cream, aerosol foam or gel.

If possible, the reduced colorant preparation is to be stabilized in the reduced form and protected from atmospheric oxygen. This can be accomplished by the use of appropriate packaging, protective gas and addition of protective and/or stabilizing substances or by esterification (so-called leuko vat dye esters prepared with chlorosulfonic acid). Protective or stabilizing compounds are, for example, cationic compounds, for example polymers, surfactants or metal ions, for example zinc ions.

To achieve a uniformly intense coloration of the hair, it is advantageous to protect the reduced form of the dye from atmospheric oxygen even during the dyeing process. This can be done, for example, by placing on the moist hair a plastic cap with valve openings distributed over its entire surface. In this manner, it is possible to “spray” under the cap through the 5 to 10 valves a relatively fluid dye mixture and then thoroughly distribute it from the outside and massage it in. It is also possible to use a plastic cap with small openings to introduce, with the aid of a filling nozzle, an aerosol foam under the cover, place the foam onto the hair and distribute it by massaging.

After the colorant has acted for about 1 to 60 minutes and preferably for 5 to 30 minutes at about 15 to 60° C. and preferably at 20 to 45° C., the coloration is “developed” oxidatively. Suitable oxidants for developing the coloration are besides simple air oxidation particularly hydrogen peroxide or the compounds of addition thereof to urea, melamine or sodium borate in the form of a 1 to 12% and preferably 1.5 to 6% aqueous solution. The mixing ratio of dye to oxidant depends on the concentration of the oxidant and usually amounts to about 5:1 to 1:2 and preferably 1:1, the amount of oxidant in the resulting mixture of dye and oxidant preferably being from 0.5 to 8 weight percent and particularly from 1 to 4 weight percent. The oxidant is allowed to act until the dye is once again in its insoluble pigment form (usually for about 2 to 5 minutes) and is thus fixed on the hair in washing-resistant manner. The treatment time can, of course, be extended depending on the degree of brightening desired. If the natural pigment is to be brightened by more than two brightening degrees, it is also possible to use a persulfate or a mixture of a persulfate and hydrogen peroxide or an addition compound thereof. Alkaline persulfate/peroxide preparations are preferably used in this case.

Following the dyeing procedure, the hair is rinsed with water and dried. Optionally, the hair can be additionally be washed with a shampoo and possibly post-rinsed with an acidic conditioner. The hair is then dried.

The following examples will explain the subject matter of the invention in greater detail without limiting it to these examples.

### EXAMPLES

### Example 1

### Dye Mixture

| | |
|---|---|
| 10.0 g | of propylene glycol |
| 1.0 g | of indigo |
| 1.0 g | of acetoin |
| 12.0 g | of sodium hydroxide (10% aqueous solution) |
| 3.0 g | of Polyquaternium-7 (8% aqueous solution) |
| 65.0 g | of water |

The afore-said substances were mixed and allowed to stand under argon for 2 hours at 40° C. Then, the pH was adjusted to 9.6 with 4.0 g of ammonia (25% aqueous solution) and 4.0 g of lactic acid (90% aqueous solution).

10 g of the mixture obtained in this manner was then applied to medium-blond natural hair. The hair was then covered with a plastic cap or plastic foil. After a treatment time of 30 min at 40° C., 10 g of a 6% aqueous hydrogen peroxide emulsion was massaged in and allowed to act for 2 minutes. The hair was then rinsed with water, washed with a shampoo, rinsed with an acidic conditioner and finally dried.

The hair assumed a uniform blue-black color.

### Example 2

### Dye Mixture

| | |
|---|---|
| 20.00 g | of propylene glycol |
| 1.00 g | of indigo |
| 1.00 g | of acetoin |
| 12.00 g | of sodium hydroxide (10% aqueous solution) |
| 0.25 g | of Polyquaternium-4 |
| 57.75 g | of water |

The afore-said substances were mixed and allowed to stand under argon for 2 hours at 40° C. The pH was then adjusted to 9.6 with 4 g of ammonia (25% aqueous solution) and 4.0 g of lactic acid (90% aqueous solution).

10 g of the mixture obtained in this manner was mixed with 5 g of a cream of the following composition by stirring

| | | |
|---|---|---|
| 10.0 | g | of lauryl ether sulfate (28% aqueous solution) |
| 11.0 | g | of cetylstearyl alcohol |
| to 100.0 | g | water |

The dye-containing cream was applied to bleached hair strands and allowed to act for 30 min at 40° C. with exclusion of oxygen. Then, 15 g of a 6% hydrogen peroxide emulsion was massaged in. After a treatment period of 2 minutes, the hair was rinsed with water. After an additional rinsing with an acidic conditioner, the hair was dried.

The hair assumed a uniform deep-blue color.

### Example 3

### Dye Mixture

| | |
|---|---|
| 10.00 g | of propylene glycol |
| 1.00 g | indigo |
| 12.00 g | of sodium hydroxide (10% aqueous solution) |
| 1.00 g | of hydroxyacetone |
| 0.65 g | of Polyquaternium-16 (40% aqueous solution) |
| 67.35 g | of water |

The afore-said substances were mixed and allowed to stand under argon for 2 hours at 40° C. The pH was then adjusted to 9.6 with 4.0 g of ammonia (25% aqueous solution) and 4.0 g of lactic acid (90% aqueous solution).

20 g of the mixture obtained in this manner was applied to light-brown natural hair. The hair was then covered with a plastic cap or plastic foil. After a treatment time of 30 min at 40° C., 20 g of a 6% aqueous hydrogen peroxide emulsion was massaged in and allowed to act for 2 minutes. The hair was then rinsed with water, then with an acidic conditioner and finally dried. The hair assumed a uniform blue-black color.

### Example 4

### Dye Mixture

| | |
|---|---|
| 10.0 g | of propylene glycol |
| 1.0 g | of indirubin |
| 12.0 g | of sodium hydroxide (10% aqueous solution) |
| 3.0 g | of Polyquaternium-7 (8% aqueous solution) |
| 1.0 g | of acetoin |
| 65.0 g | of water |

The afore-said substances were mixed and allowed to stand under argon for 2 hours at 40° C. The pH was then adjusted to 9.6 with 4.0 g of ammonia (25% aqueous solution) and 4.0 g of lactic acid (90% aqueous solution).

15 g of the mixture obtained in this manner was applied to bleached natural hair. The hair was then covered with a plastic cap or plastic foil. After a treatment time of 30 min at 40° C., 15 g of a 6% aqueous hydrogen peroxide emulsion was massaged in and allowed to act for 2 minutes. The hair was rinsed with water, then with an acidic conditioner and finally dried. The hair assumed a uniform red-violet color.

### Example 5

### Dye Mixture

| | |
|---|---|
| 10.0 g | of propylene glycol |
| 1.0 g | of indigo |
| 12.0 g | of sodium hydroxide (10% aqueous solution) |
| 3.0 g | of Polyquaternium-7 (8% aqueous solution) |
| 1.0 g | of acetoin |
| 62.6 g | of water |

The afore-said substances were mixed and allowed to stand under argon for 2 hours at 40° C. The pH was then adjusted to 9.6 with 4.0 g of ammonia (25% aqueous solution) and 4.0 g of lactic acid (90% aqueous solution) and the oxidative dye precursors (1.5 g of 1-hydroxyethyl-4,5-diaminopyrazole sulfate, 0.8 g of 4-amino-2-hydroxytoluene and 0.1 g of 2,6-diamino-3-{[(pyridin-3-yl)azo]pyridine} were added.

20 g of the mixture obtained in this manner was then applied to natural dark-blond hair. The hair was then covered with a plastic cap or plastic foil. After a treatment time of 15 min at 40° C., 30 g of a 6% aqueous hydrogen peroxide emulsion was massaged in and allowed to act for 10 minutes. The hair was then rinsed with water, then with an acidic conditioner and finally dried. The hair assumed a uniform red-brown color.

### Example 6

### Dye Mixture

| | |
|---|---|
| 10.0 g | of propylene glycol |
| 1.0 g | of 6,6′-dibromoindigo |
| 12.0 g | of sodium hydroxide (10% aqueous solution) |
| 3.0 g | of Polyquaternium-7 (8% aqueous solution) |
| 1.0 g | of acetoin |
| 65.0 g | of water |

The afore-said substances were mixed and allowed to stand under argon for 2 hours at 40° C. The pH was then adjusted to 9.6 with 4.0 g of ammonia (25% aqueous solution) and 4.0 g of lactic acid (90% aqueous solution).

10 g of the mixture obtained in this manner was applied to bleached natural hair. The hair was then covered with a plastic cap or plastic foil. After a treatment time of 30 min at 40° C., 10 g of a 6% aqueous hydrogen peroxide emulsion was massaged in and allowed to act for 2 minutes. The hair was rinsed with water, then with an acidic conditioner and finally dried. The hair assumed a uniform pink color.

Unless otherwise indicated, all percentages given in the present patent application are by weight.

1-13. (canceled)

14. An agent for coloring hair, said agent comprising at least one indigoid vat dye, at least one cationic compound, and, as reducing agent, at least one compound that forms an enediol in an alkaline medium.

15. The agent as defined in claim 14, wherein said at least one compound that forms said enediol in said alkaline medium is selected from the group consisting of monohydroxyacetone, dihydroxyacetone, acetoin, glutaroin, adipoin, glycol aldehyde, benzoin, 2,3-dihydroxyacrylaldehyde, and cyclopentadiolone.

16. The agent as defined in claim 14, containing said at least one compound that forms said enediol in said alkaline medium in an equimolar amount or in an up to 50-fold excess, based on said at least one indigoid vat dye.

17. The agent as defined in claim 14, wherein said at least one indigoid vat dye is selected from the group consisting of indigo, indirubin, 6,6′-dibromoindigo, 5,5′,7,7′-tetrabromoindigo, 4,4′,7,7′-tetrachloroindigo, thioindigo, 6,6′-dichloro-4,4′-dimethylthioindigo, 5,5′-dichloro-7,7′-dimethylthioindigo, 4,4′,7,7′-tetramethyl-thioindigo, and mixtures thereof.

18. The agent as defined in claim 14, containing from 0.01 to 10 weight percent of said at least one indigoid vat dye.

19. The agent as defined in claim 14, containing from 0.001 to 5 weight percent of said at least one cationic compound, and wherein said at least one cationic compound is a cationic polymer.

20. The agent as defined in claim 14, wherein said at least one cationic compound is selected from the group consisting of Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium-44, Polyquaternium-46, Polyquaternium-47, Polyquaternium-51, Polyquaternium-55, Polyquaternium-57, hydroxypropylguar-hydroxypropyltrimethylammonium chloride, guar-hydroxypropyl trimethylammonium chloride, Quaternium-80, and mixtures thereof.

21. The agent as defined in claim 14, further comprising developers and/or couplers and/or further comprising natural or synthetic direct dyes.

22. A ready-to-use agent for coloring hair, said ready-to-use agent having a pH of 4 to 11 and containing a cationic compound and a reduced indigoid vat dye, and wherein said reduced indigoid vat dye is formed by reducing an indigoid vat dye with a compound that forms an enediol in an alkaline medium in the presence of the cationic compound.

23. The ready-to-use agent as defined in claim 22, wherein said compound that forms said enediol in said alkaline medium is selected from the group consisting of monohydroxyacetone, dihydroxyacetone, acetoin, glutaroin, adipoin, glycol aldehyde, benzoin, 2,3-dihydroxyacrylaldehyde, and cyclopentadiolone.

24. The ready-to-use agent as defined in claim 22, containing said compound that forms said enediol in said alkaline medium in an equimolar amount or in an up to 50-fold excess, based on said indigoid vat dye.

25. The ready-to-use agent as defined in claim 22, wherein said indigoid vat dye is selected from the group consisting of indigo, indirubin, 6,6′-dibromoindigo, 5,5′,7,7′-tetra-bromoindigo, 4,4′,7,7′-tetrachloroindigo, thioindigo, 6,6′-dichloro-4,4′-dimethyl-thioindigo, 5,5′-dichloro-7,7′-dimethylthioindigo, 4,4′,7,7′-tetramethyl-thioindigo, and mixtures thereof.

26. The ready-to-use agent as defined in claim 22, containing from 0.01 to 10 weight percent of said vat dye, based on said indigoid vat dye.

27. The ready-to-use agent as defined in claim 22, containing from 0.001 to 5 weight percent of said cationic compound, and wherein said cationic compound is a cationic polymer.

28. The ready-to-use agent as defined in claim 22, wherein said cationic compound is selected from the group consisting of Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium-44, Polyquaternium-46, Polyquaternium-47, Polyquaternium-51, Polyquaternium-55, Polyquaternium-57, hydroxypropylguar-hydroxypropyltrimethylammonium chloride, guar-hydroxypropyl trimethylammonium chloride, Quaternium-80, and mixtures thereof.

29. The ready-to-use agent as defined in claim 22, further comprising developers and/or couplers and/or further comprising natural or synthetic direct dyes.

30. A method of coloring hair at a pH of 4 to 11 with at least one pre-reduced indigoid vat dye, wherein said at least one pre-reduced indigoid vat dye is formed by reducing an indigoid vat dye with a compound that forms an enediol in an alkaline medium in the presence of a cationic compound.

31. A method of color hair, said method comprising the steps of:
a) reducing at least one indigoid vat dye with a compound that forms an enediol in an alkaline medium having a pH of 10 to 13 in the presence of a cationic compound to form at least one pre-reduced indigoid vat dye;
b) after the reducing step a), applying said at least one pre-reduced indigoid vat dye to the hair at a pH of 4 to 11;
c) allowing said at least one pre-reduced indigoid vat dye to act on the hair for from 1 to 60 minutes at 15 to 60° C.; and
d) after the allowing of step c), re-oxidizing said at least one indigoid vat dye with atmospheric oxygen or an oxidant to form an insoluble pigment.

32. The method as defined in claim 31, wherein said oxidant is hydrogen peroxide or an addition compound of said hydrogen peroxide with urea, melamine, or borate; a persulfate; or a mixture thereof.

33. The method as defined in claim 31, further comprising protecting the hair from said atmospheric oxygen during said allowing of said at least one pre-reduced indigoid vat dye to act on the hair.

34. The method as defined in claim 31, wherein said at least one indigoid vat dye is selected from the group consisting of indigo, indirubin, 6,6′-dibromoindigo, 5,5′,7,7′-tetrabromoindigo, 4,4′,7,7′-tetrachloroindigo, thioindigo, 6,6′-dichloro-4,4′-dimethylthioindigo, 5,5′-dichloro-7,7′-dimethylthioindigo, 4,4′,7,7′-tetramethyl-thioindigo, and mixtures thereof.

35. The method as defined in claim 31, wherein said compound that forms said enediol in said alkaline medium is selected from the group consisting of monohydroxyacetone, dihydroxyacetone, acetoin, glutaroin, adipoin, glycol aldehyde, benzoin, 2,3-dihydroxyacrylaldehyde, and cyclopentadiolone.

36. The method as defined in claim 31, wherein said cationic compound is selected from the group consisting of Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium-44, Polyquaternium-46, Polyquaternium-47, Polyquaternium-51, Polyquaternium-55, Polyquaternium-57, hydroxypropylguar-hydroxypropyltrimethylammonium chloride, guar-hydroxypropyl trimethylammonium chloride, Quaternium-80, and mixtures thereof.
